(19) United States
(12) Patent Application Publication
(10) Pub. No.: US 2023/0134228 A1
(43) Pub. Date: May 4, 2023

(54) SYNERGISTIC HERBAL COMPOSITION AS A BROAD-SPECTRUM PROPHYLACTIC MAJOR AND METHOD TO PREPARE THE SAME

(71) Applicants: Pawan Kumar SINGH, Pune (IN); Sarita SINGH, Pune (IN); Vaishali Hanmantrao GAIKWAD, Pune (IN); Aditi Hanmantrao GAIKWAD, Pune (IN)

(72) Inventors: Pawan Kumar SINGH, Pune (IN); Sarita SINGH, Pune (IN); Vaishali Hanmantrao GAIKWAD, Pune (IN); Aditi Hanmantrao GAIKWAD, Pune (IN)

(21) Appl. No.: 17/915,476

(22) PCT Filed: Mar. 30, 2021
(86) PCT No.: PCT/IB2021/052649

§ 371 Date: Sep. 28, 2022

(30) Foreign Application Priority Data
Mar. 30, 2020 (IN) 202021013797

(51) Int. Cl. A61K 36/58; A61K 36/752; A61K 36/899; A61K 36/53; A61K 36/534; A61K 36/324; A61K 36/61; A61K 36/23; A61K 36/71; A61K 36/81; A61K 36/185; A61K 36/8945; A61K 36/48; A61K 36/82; A61K 36/9068; A61K 36/484; A61K 36/54; A61K 36/889; A61K 47/10; A61K 47/26

(52) U.S. Cl. CPC A61K 36/58; A61K 36/752; A61K 36/899; A61K 36/53; A61K 36/534; A61K 36/324; A61K 36/61; A61K 36/23; A61K 36/71; A61K 36/81; A61K 36/185; A61K 36/8945; A61K 36/48; A61K 36/82; A61K 36/9068; A61K 36/484; A61K 36/54; A61K 36/889; A61K 47/10; A61K 47/26; A61K 2236/333; A61K 2236/17

(57) ABSTRACT

Disclosed herein are formulations of a poly-herbal synergistic composition which exhibit marked immunomodulatory, anticancer, antiviral and anti-inflammatory properties. Also disclosed are the methods of preparing and using the same.

### CROSS REFERENCES TO RELATED APPLICATIONS

This complete specification is filed further to application for patent No. 202021013797 filed on Mar. 30, 2020 with provisional specification, the contents of which are incorporated herein in their entirety, by reference.

### FIELD OF THE INVENTION

This invention belongs to the field of herbal medicines. More particularly, the present invention outlines an inventive poly-herbal synergistic composition having significant anticancer, antiviral, anti-inflammatory and immunomodulatory properties. Furthermore, the present invention outlines the methods for preparation and use of said poly-herbal composition.

### DEFINITIONS AND INTERPRETATIONS

Before undertaking the detailed description of the invention below, it may be advantageous to set forth definitions of certain words or phrases used throughout this patent document: the terms “include” and “comprise,” as well as derivatives thereof, mean inclusion without limitation; the term “or” is inclusive, meaning and/or; the phrases “associated with” and “associated therewith,” as well as derivatives thereof, may mean to include, be included within, interconnect, with, contain, be contained within, connect to or with, couple to or with, be communicable with, cooperate with, interleave, juxtapose, be proximate to, be bound to or with, have, have a property of, or the like; and the term “Herb” refers generally a material of plant origin as recognized by practitioners of both traditional and modern herbal medicine; “IgG” refers Immunoglobulin G; “AML” refers Acute Myeloid Leukemia; “HDF” refers Human Dermal Fibroblast; “MTT” refers the standard laboratory colorimetric assay for assessing cell metabolic activity; “PBS” shall mean phosphate buffered saline; as to the formulations, CH & CF (formulation 1) is for anti-cancer properties and SH (Formulation 2) is mentioned for immunity and antiviral action.

### BACKGROUND OF THE INVENTION AND DESCRIPTION OF RELATED ART

Health concerns continue to rise at an alarming rate all over the world. Aggressive adoption of urban and more specifically sedentary lifestyles, weakened immunities that warrant susceptibility to infectious diseases, advent of new-world diseases, and overall drug resistance that the old-world disease-causing agents have managed to achieve in recent times are some of the major reasons behind said concerns.

It would hence be desirable to have some effective means to prevent, contain, and/or treat the diseases that continue to plague and ravage, or otherwise compromise incumbency of a healthy populace. It would be necessary that such means are characterized in having the following virtues—
a) Broad-spectrum efficacy;
b) Prophylactic/therapeutic activity at minimal dosage/application;
c) Easy, cost-effective production and usage;
d) Long shelf stability;
e) No side/adverse effects on subjects on short as well as long term administration; and
f) No ill-effects on the environment as incidental to sourcing of herbs, production process and/or processes for disposal.

Medicinal plants and plant derived products have been recognized as major resources for alternative therapy for several diseases since long time. Though, information based on experience about such medicinal plants is extensively available in traditional system of Indian medicine like Ayurveda, there is dire need for scientific validation of such claims and standardization before recommendation as any form of therapy.

A survey of literature reveals a few attempts being undertaken in this direction. For example, patent application no. U.S. Pat. No. 8,163,312B contains information of polyherbal formulation for diabetes and associated complications. The polyherbal formulation contains Emblica officinalis, Terminalia chebula, Centella asiatica, Mangifera Indica, Curcuma longa, etc. similarly patent application no. US20160184381A1 educate us about anticancer properties of β-Sitosterol in various carcinoma cell lines and its fortification using other phytosterols such as Boswellic acid, Betulinic acid, Epigallacotechin-3-gallate etc.

On similar path patent application no. U.S. Pat. No. 6,780,441 B2 a polyherbal formulation of eleven herbs such as Tecoma, Ficus, Boerhavia, Withania, Moringa etc. for treatment of cell carcinoma, tumors and growth prevention. On parallel track patent application no. WO1994018993A1 contains information for a therapeutic composition of herbal composition useful in treatment of diverse range of diseases such as cancer, viral infections (AIDS, Epstein Barr Syndrome) as well useful health tonic. This formulation contains synergistic combination of Cinnamomum, Syzygium, Allium, Cyperus etc.

Plant extracts have reports for antiviral activity, immunomodulatory and anticancer activity. Various other plants have been used as immunomodulatory agents include Tulsi, Kaalmegha, Kaanchana, Safedmusali, Shallaki, Tea, lavanga etc. (Dinesh Kumar et al. 2012 Journal of Microbiology, Immunology and Infection 45(3)165-184, K Thangadurai et al 2018 International Journal of Herbal Medicine 6(6) 10-14). Chatterje et al have shown Ocimum sanctum (100 mg) extract along with Tinospora cordifolia, Withania somenifera and Emblica officinalis increased the CD4 cell count and decrease viral load in HIV patients.

Kawaii et al evaluated at in vivo activity of Citrus juice at 100 μg/ml in various cell lines suing alamar blue assay found 48% to 87% inhibition, similarly C. limon showed IC50 at 1.4% v/v concentration using Trypan Blue exclusion method (Fernandez-Bedmer et al). Ginger extract and its active components were evaluated for the anticancer activity by Brahmbhatt M et al, these active showed IC50 ranging from 1.12 to 22.07 μg/ml.

Nigella sativa contains Thymoquinone which is known to enhance helper and Cytotoxic T cells and decrease viral proteins such as integrase, protease and RNA Polymerase-II and helpful in immunomodulation and antiviral action. Licorice contain Glycyrrhizin (GL), 18β-glycyrrhetinic acid (GA), liquiritigenin (LTG), licochalcone A (LCA), licochalcone E (LCE) and glabridin (GLD) known for their antiviral activity. These triterpenes of are also known for their anti-inflammatory, anti-allergy and anti-cancer properties.

Patent WO2014142645A1 claims an antiviral medicament which comprises of a natural flavonoid called querctin, similarly US Patent US20030165579 claims anticancer activity of extract of Euphorbia antiquorum in treatment in liver cancer and colon cancer. U.S. Pat. No. 7,201,298 claims extract of orange peel for prevention and treatment of cancer. Similarly U.S. Pat. No. 7,709,031 showed antiangiogenic activity of isolated phytochemical Gallic acid from Chinese blackberry extract.

Cinnamon bark contains cinnamaldehyde, cinnamic acid, cinnamyl alcohol, coumarin, and eugenol as the major components which are proven for anti-oxidant, immunomodulatory, anti-inflammatory and anti-tumorgenic activities. Tea contains biological compounds such as flavonoids, theaflavins, phenolic acid, gallic acid and β-carotene. Tea extracts has been proven for pharmacological effects such as cardio protector, antioxidant and anticancer. Peppermint oil contains menthol, limonene, carvoneetc as major components and is known to have the antibacterial, antiviral, anti-allergic and anti-inflammatory action.

Plant extracts such as Azadirachta indica, Hedera helix, Pelargonium sidoides, Galla chinensis, Juglans regia, Schisandra chinensis has shown inflammation inhibition actions in lung infections (Kim et al 2017 BiomolTher 25(2)91-104. Various plants such as Dictyophora indusiata, Vigna vexillata have been studied for antioxidant and anti-inflammatory activity (Chinese Patent CN102512353A, Yann-LiiLeu et al 2012 Int J MolSci 13(8) 9754-9768).

However, prior art concerns mostly with single plant extracts. And in cases wherein more than one herbal source is used, the choice of ingredients is based solely on achieving an aggregation of their individual properties. Hence, the art misses a lot on synergistic studies and possibility of significantly enhanced drug efficacy and/or improvement of other parameters related thereto. Due to this, prior art does not list a single effective solution embracing all considerations mentioned hereinabove, thus preserving an acute necessity-to-invent for the applicants named herein. And as a result of their focused research, the applicants named herein have come up with a novel solution for resolving all needs of the art once and for all. The virtual absence of any reliable means to achieve the aforesaid wants of art form the background, hence incentive, for this invention.

State-of-art therefore, does not list a single effective solution embracing all considerations mentioned hereinabove, thus preserving an acute necessity-to-invent for the present inventor/s who, as result of focused research, has come up with novel solutions for resolving all needs once and for all. Work of the presently named inventor/s, specifically directed against the technical problems recited hereinabove and currently part of the public domain including earlier filed patent applications, is neither expressly nor impliedly admitted as prior art against the present disclosures.

A better understanding of the objects, advantages, features, properties and relationships of the present invention will be obtained from the following detailed description which sets forth an illustrative yet-preferred embodiment.

### OBJECTIVES OF THE PRESENT INVENTION

Principally, a general objective of the present invention is to assess disabilities and shortcomings inherent to known systems comprising state of the art and develop new systems incorporating all available advantages of known art and none of its disadvantages.

The present invention is identified in addressing at least all major deficiencies of prior art discussed in the foregoing section by effectively achieving the following objectives—

It is a primary objective to establish a poly-herbal composition having marked antiviral, immunomodulatory, anticancer and anti-inflammatory properties.

It is another objective further to the aforesaid objective(s) that said poly-herbal composition is safe for human consumption without side effects.

The manner in which the above objectives are achieved, together with other objects and advantages which will become subsequently apparent, reside in the detailed description set forth below in reference to the accompanying drawings and furthermore specifically outlined in the independent claims 1. Other advantageous embodiments of the invention are specified in the dependent claims.

### BRIEF DESCRIPTION OF DRAWINGS

The present invention is explained herein under with reference to the following drawings, in which:

FIG. 1 is a graph showing size distribution of particles in the formulation which have been converted to nano-sized form.

FIG. 2A is a graph showing activity of a first embodiment of the poly-herbal composition proposed herein, against THP1 cell line.

FIG. 2B is a graph showing activity of a first embodiment of the poly-herbal composition proposed herein against HL-60 cell line.

FIG. 3A is a graph showing activity of a first embodiment of the poly-herbal composition proposed herein, against 4T1 cell line.

FIG. 3B is a graph showing activity of a first embodiment of the poly-herbal composition proposed herein against MDA-MB-231 cell line.

FIG. 4 is a graph showing activity of a first embodiment of the poly-herbal composition proposed herein against PC3 cell line.

FIG. 5 is a graph showing activity of a first embodiment of the poly-herbal composition proposed herein against NIH/3T3 cell line.

FIG. 6A includes a compilation of photographs of the wound healing assay undertaken using a first embodiment of the poly-herbal composition proposed herein in MDA-MB-231 cell line model.

FIG. 6B includes a graphical representation of data captured during the trial shown in FIG. 6A.

FIG. 7 is a graph showcasing results of delayed type hypersensitivity assay undertaken using a first embodiment of the poly-herbal composition proposed herein in rat model.

FIG. 8 is a graph showcasing immuno stimulatory effect of a first embodiment of the poly-herbal composition proposed herein against cyclophosphamide induced immuno suppression in mouse model.

FIG. 9 is a graph showcasing enhancement of phagocytic index by a first embodiment of the poly-herbal composition proposed herein against clearance of carbon (Indian ink) in mouse model.

FIG. 10Ais a graph showcasing enhancement of serum levels of TNF-α by a first embodiment of the poly-herbal composition proposed herein in mouse model.

FIG. 10B is a graph showcasing enhancement of serum levels of IgG by a first embodiment of the poly-herbal composition proposed herein in mouse model.

FIG. 11(A to H) are a series of micro-photographs showcasing results of histopathological studies undertaken for a first embodiment of the poly-herbal composition proposed herein in mouse model administered in a mouse model.

FIG. 12 contains a series of micro-photographs showcasing results of confocal microscopy analysis for assessing actin depolymerisation in MDA-MB-231 cell line by upon administration of a first embodiment of the poly-herbal composition proposed herein.

FIG. 13(A to E) are a series of graphs showcasing results of cell cycle analysis through Flow cytometry cell cyclein MDA-MB-231 cell line by upon administration of a first embodiment of the poly-herbal composition proposed herein.

FIG. 13F includes a graphical representation of data captured during the trial shown in FIG. 13(A to E).

FIG. 14 is a graph showing effect of a second embodiment of the poly-herbal composition proposed herein, on antibody titer assay in SRBC immunized rats.

FIG. 15 is a graph showcasing results of delayed type hypersensitivity assay undertaken using the second embodiment of the poly-herbal composition proposed herein in rat model.

FIG. 16 is a graph showcasing immunostimulatory effect of the second embodiment of the poly-herbal composition proposed herein against cyclophosphamide induced immunosuppression in mouse model.

FIG. 17 is a graph showcasing enhancement of phagocytic index by the second embodiment of the poly-herbal composition proposed herein against clearance of carbon (Indian ink) in mouse model.

FIG. 18Ais a graph showcasing enhancement of serum levels of IgG by the second embodiment of the poly-herbal composition proposed herein in mouse model.

FIG. 18B is a graph showcasing enhancement of serum levels of TNF-α by the second embodiment of the poly-herbal composition proposed herein in mouse model.

FIG. 19(A to G) are a series of micro-photographs showcasing results of histopathological studies undertaken for the second embodiment of the poly-herbal composition proposed herein in mouse model administered in a mouse model.

The above drawings are illustrative of particular examples of the present invention but are not intended to limit the scope thereof. The drawings are not to scale (unless so stated) and are intended for use solely in conjunction with their explanations in the following detailed description. In above drawings, wherever possible, the same references and symbols have been used throughout to refer to the same or similar parts. Though numbering has been introduced to demarcate reference to specific components in relation to such references being made in different sections of this specification, all components are not shown or numbered in each drawing to avoid obscuring the invention proposed.

Attention of the reader is now requested to the detailed description to follow which narrates a preferred embodiment of the present invention and such other ways in which principles of the invention may be employed without parting from the essence of the invention claimed herein.

### STATEMENT OF THE PRESENT INVENTION

Formulations of a poly-herbal synergistic composition, and methods of preparing and using the same, are proposed herein. Said formulations exhibit marked immunomodulatory, anticancer and anti-inflammatory properties. The composition comprises Azardiracta indica; Citrus sinensis; Zea mays; Origanum vulgare; Mentha piperita; Boswellia serrata; Citrus bergamia; Melaleuca alternifloia; Eucalyptus globules; Coriandrum sativum; Rosemarinus officinalis; Carumc opticum; Nigella sativa; Cymbopogon citrates; Saccharum officinarum; Ipomoea aquatic; Eleusine coracana; Biophytum sensitivum; Dioscorea bulbifera; Bambusa bambosa; Taverniera cuneifolia; Camellia sinensis; Vigna vexillata; Delinea indica; Zingiber officinalis; Glycyrrhiza glabra; Ocimum sanctum; Lavandulla sp; Mentha arvensis; Syzygium aromaticum; Cinnamon spp. selected between Cinnamon verum and Cinnamon zeylanicum and Cocos nucifera.

### DETAILED DESCRIPTION

Generally speaking, the present invention is directed at absorbing all advantages of prior art while overcoming, and not imbibing, any of its shortfalls. More specifically, but without restriction to the particular embodiments hereinafter described in accordance with the best mode of practice, This invention provides a broad-spectrum poly-herbal synergistic composition for the effective prophylaxis, containment, and/or treatment of cancer and other indications.

This invention is based on the approach that combining multiple plants or plant extracts can enhance the pharmacological efficacy of their individual medicinal properties via synergistic action. Furthermore, the applicants hereof are of the belief that use of the poly-herbal synergistic composition proposed herein shall assuredly help in not only curbing the incidence of cancer and other indications but also promote overall health and innate immunity of individuals which are critical for preventing disease and/or the development of allied disease-related complications.

Reference is made now to certain examples, which showcase foundational aspects of the present invention. These examples are exemplary and not intended to be limiting. Accordingly, the broad-spectrum poly herbal synergistic composition of the present invention is typified in comprising a list of ingredients identified as per at least one of the Tables 1 below.

| Sr. | | | | Part of | |
|---|---|---|---|---|---|
| No. | Common name | Botanical name | Range % | plant used | Source |
| 1. | Neem | Azardiracta indica | 0.01 to 15 | Leaf and fruit | Procured from |
| 2. | Sweet orange | Citrus sinensis | 0.01 to 10 | Leaf and fruit | various local |
| 3. | Corn | Zea mays | 0.15 to 10 | seeds | vendors/ |
| 4. | Oregano | Origanum vulgare | 0.01 to 10 | Whole Plant | open market |
| 5. | Peppermint | Mentha piperita | 0.01 to 15 | Whole Plant | |
| 6. | Frankinsence | Boswellia serrata | 0.01 to 12 | Extract and | |
| | | | | oleo cum resin | |
| 7. | Bergamout | Citrus bergamia | 0.01 to 10 | Fruit and leaves | |
| 8. | Tea tree | Melaleuca | 0.01 to 15 | Aerial parts | |
| | | alternifloia | | | |
| 9. | Eucalyptus | Eucalyptus | 0.01 to 15 | Aerial parts | |
| | | globulus | | | |
| 10. | Coriander | Coriandrum | 0.01 to 15 | Whole plants | |
| | | sativum | | | |
| 11. | Rosemary | Rosemarinus | 0.01 to 15 | Aerial parts | |
| | | officinalis | | | |
| 12. | Thyme | Carum copticum | 0.01 to 15 | Whole plants | |
| 13. | Black Seed | Nigella sativa | 0.01 to 15 | Fruit and seed | |
| 14. | Lemongrass | Cymbopogon | 0.01 to 10 | Whole plants | |
| | | citrates | | | |
| 15. | Sugarcane | Saccharum | 0.15 to 15 | Aerial parts | |
| | | officinarum | | | |
| 16. | Swamp | Ipomoea aquatic | 0.02 to 5.0 | Aerial parts | |
| | morning glory | | | | |
| 17. | Ragi | Eleusine coracana | 0.02 to 5.0 | seeds | |
| 18. | Little tree | Biophytum | 0.02 to 5.0 | Arial parts | |
| | plant | sensitivum | | | |
| 19. | Air yam | Dioscore abulbifera | 0.02 to 5.0 | Rhizome | |
| 20. | Thorny bamboo | Bambusa bambosa | 0.02 to 5.0 | Aerial parts | |
| 21. | Jethmad | Taverniera | 0.02 to 5.0 | Aerial parts | |
| | | cuneifolia | | | |
| 22. | Tea | Camellia sinensis | 0.02 to 5.0 | Aerial parts | |
| 23. | Wild cow pea | Vigna vexillata | 0.02 to 5.0 | Aerial parts | |
| 24. | Elephant apple | Delinea indica | 0.02 to 5.0 | Aerial parts | |
| 25. | Zinger | Zingiber officinalis | 0.02 to 5.0 | Roots | |
| 26. | Mulethi | Glycyrrhiza glabra | 0.1 to 15. | Extract/whole | |
| | | | | plant | |
| 27. | Tulsi | Ocimum sanctum | 0.02 to 4.0 | Extract/whole | |
| | | | | plant | |
| 28. | Lavender | Lavandulla sp | 0.02 to 5.0 | Extract/whole | |
| | | | | plant/ | |
| | | | | Aerial parts | |
| 29. | Spearmint | Mentha arvensis | 0.01 to 10 | Extract/whole | |
| | | | | plant/ | |
| | | | | Aerial parts | |
| 30. | Clove | Syzygium | 0.01 to 10 | Fruits/flowers | |
| | | aromaticum | | | |
| 31. | Cinnamon | Cinnamon verum | 0.01 to 10 | Aerial/bark | |
| | | Or Cinnamon | | | |
| | | zeylanicum | | | |
| 32. | Coconut | Cocos nucifera | 0.02 to 5.0 | Fruits/extract | |

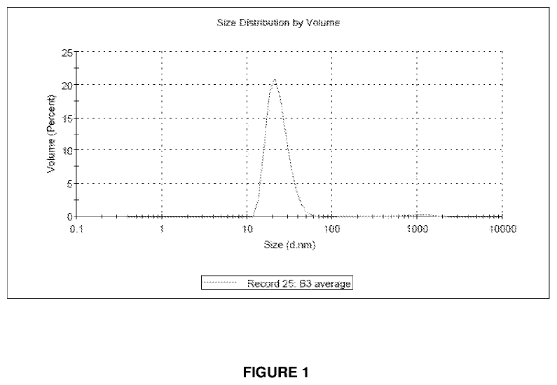

A model preparatory process for synthesis of the broad-spectrum poly-herbal synergistic composition of the present invention may be appreciated from the performance, in serial, of the following combination of inventive and known steps—
a) Procurement of raw material.
b) Shade drying of herbs for 3-7 days.
c) Powdering of herbs.
d) Sieving of herbs using different mesh size 50-500.
e) Soaking of herbs separately in solvent(mainly Ethyl alcohol and water, or may be polar and/or non polar organic solvents, alcohols etc.) in ratio 1:25 for 24-72 hrs.
f) Boiling of herbs at 80-100° C. for 60-180 minutes.
g) Concentrating and purification and active extract preparation.
h) Mixing all the active concentrates at 200-1000 RPM.
i) Sonicating the mixture for 15-60 minutes for conversion to nano-sized particles for better efficacy (size distribution shown in FIG. 1)
j) Leaving the mixture for maturation for 24-72 hrs.
k) Again sonicating the mixture for 15-60 minutes
l) Addition of standard formulation agents such as Tween 80, PEG, Sugar for making stable formulations

The novel and inventive broad-spectrum poly-herbal synergistic composition of the present invention reached as per the foregoing narration has been subjected to extensive characterization and trials by the applicants named herein in in-vitro breast, prostate, blood cancer, AML trials, where it has been observed to markedly exhibit enhanced antioxidant, immuno-modulatory, anti-inflammatory, immuno-stimulatory (indicated by increased platelet count, increased IgG levels and enhanced phagocytic index), and antibiotic properties sufficient to warrant the promise of combating ill-effects of oxidative stress on body cells as well as an effective and hitherto unreported broad-spectrum applicability, without any (or insignificantly if at all) toxicity, in preventing the onslaught, delaying progression, or treatment of cancer and various other indications. The individual extracts are not having such activities, final compositions are more effective

Details of these experimental studies are provided in the narrative to follow.

A] Anti-Cancer Properties
The applicants named herein have independently validated the poly-herbal composition proposed herein, as to its efficacy against various cancer cell types such as breast cancer, colon cancer, liver cancer, blood cancer, oral cavity and pancreatic cancer.
The potential of formulations was evaluated for effectiveness on various cancer cell lines such as breast cancer (MDA-MB-231, 4T1), Leukemia Cell lines (HL-60, THP-1), Prostate Cancer (PC3) along with safety studies in NIH/3T3 cell lines.

Validation Study 1: MTT Assay on HDF Cell Line
Method: HDF cells were revived and 0.1 million cells were seeded in 96 well plate. The cells were incubated in CO₂ incubator at 37° C., 5% CO₂ overnight.
After observing the fully confluent cells under microscope the cells were treated with the poly-herbal synergistic composition of the present invention at 7 different concentrations from 0.00128 to 100 μl per well with or without PBS. The cells were incubated overnight in the presence of drug in CO₂ incubator at 37° C., 5% CO₂. After observing the cells under microscope 10 μl of 5 mg/ml MTT reagent was added in the wells and incubated for 4 hours.
Results: IC₅₀ of CH formulation on HDF cell line without PBS was found to be 0.075 μl as per the accompanying FIG. 1A. IC₅₀ of the CH formulation on HDF cell line with PBS was found to be 0.109 μL as per the accompanying FIG. 1B.

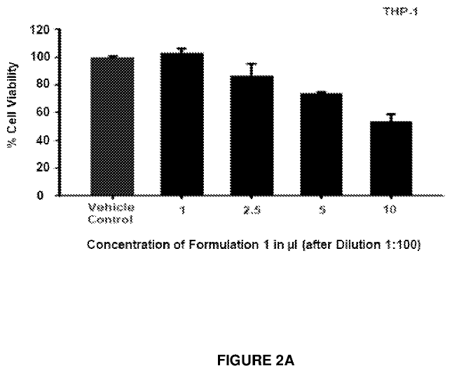

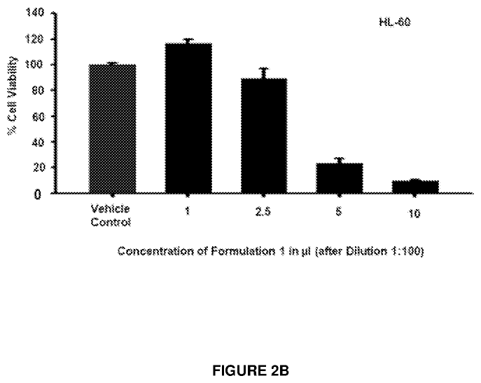

Validation Study 2: Cell Viability Assay on THP1 and HL-60 Cell Lines
Method: THP1 (Human leukemia monocytic cell line) and HL-60 (Human acute promyelocytic cell line) cells were maintained in RPMI medium containing 10% FBS, penicillin (100 U/ml) and streptomycin (100 mg/ml) under humidified atmosphere at 37° C. HL-60 cells maintained in Modified Dulbecco's medium contains 10% FBS, penicillin (100 U/ml) and streptomycin(100 mg/ml) under humidified atmosphere at 37° C. and 5% CO₂. 1×10⁴ cells (THP1 and HL-60) were seeded per well in 96 well plate followed by 24 hours of incubation.
Treatment of CH formulation were given at different concentrations from 1:100 dilution. After 48 hours incubation, MTT solution was added per well to achieve a final concentration of 0.5 mg/ml and incubated for 4 hours at 37° C. Isopropanol (solubilizing solution) was added to dissolve formazan crystals and absorbance was recorded at 570 nm on a micro plate reader (Epoch 2Microplate Reader, BioTek, USA).
Results: THP1 and HL-60 cell lines treated with CH formulation at different concentrations from 1:100 dilutions showed maximum anti-proliferative activity at 10 μl. HL-60 cells are more sensitive towards formulation as compared to THP-1 as per the accompanying FIG. 2A and FIG. 2B.

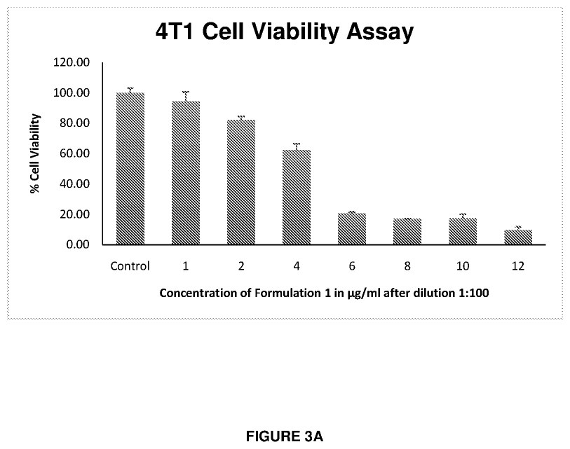

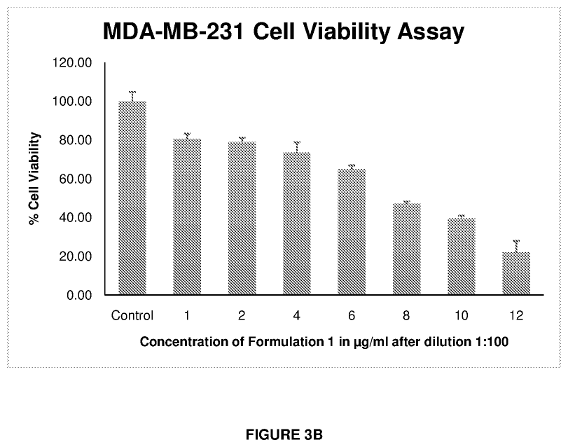

Validation Study 3: Cell Viability Assay on 4T1 and MDA-MB-231
Method: 4T1 (Mouse Breast Cancer) and MDA-MB-231 (Human Breast Cancer) cell lines were maintained in Dulbecco's medium contains 10% FBS, penicillin (100 U/ml) and streptomycin(100 mg/ml) under humidified atmosphere at 37° C. 1×10⁴ cells of 4T1 and 2×10⁴ of MDAMB-231 cells seeded per well in 96 well plate followed by 24 hours of incubation.
Treatments of CH formulation were given at different concentrations from 1:100 dilutions. After 24 hours incubation, MTT (3-(4,5-dimethylthiazol-2-yl)-2,5-diphenyltetrazoliumbromide) solution was added per well to achieve a final conc. of 0.5 mg/ml and incubated for4 hours at 37° C. Isopropanol (solubilizing solution) was added to dissolve formazan crystals and absorbance was recorded at 570 nm on a micro plate reader (Epoch 2 Microplate Reader, BioTek, USA).
Results: As per the accompanying FIG. 3A and FIG. 3B, the 4T1 and MDA-MB-231 cell lines treated with CH formulation at different concentrations from 1:100 dilutions showed maximum anti-proliferative activity at 12 μl. 4T1 cells are more sensitive towards formulation as compare to MDA-MB-231.

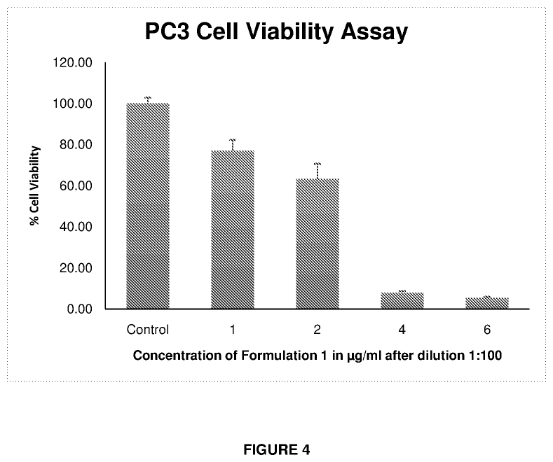

Validation Study 4: Cell Viability Assay on PC3 (Human Prostate Cancer Cell Line)
Method: PC3 (Human Prostate Cancer) cell line obtained from American Type Culture Collection (ATCC, Manassas, Va., USA) and used for this assay. PC3 maintained in F-12K medium contains 10% FBS, penicillin (100 U/ml) and streptomycin (100 mg/ml) under humidified atmosphere at 37° C. 2×10⁴ of PC3 cells seeded per well in 96 well plate followed by 24 hours of incubation.
Treatment of CH formulations were given at different concentrations from 1 to 6 μg/ml. After 24 hours incubation, MTT (3-(4,5-dimethylthiazol-2-yl)-2,5-diphenyltetrazolium bromide) solution was added per well to achieve a final conc. of 0.5 mg/ml and incubated for 4 hours at 37° C. Isopropanol (solubilizing solution) was added to dissolve formazan crystals and absorbance was recorded at 570 nm on a micro plate reader (Epoch 2 Microplate Reader, BioTek, USA).
Results: As shown in FIG. 4, the PC3 cell lines treated with CH formulation at different concentrations showed maximum anti-proliferative activity at 6 μg/ml.

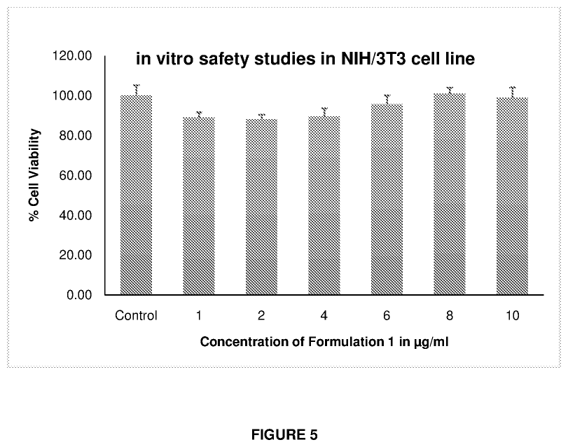

Validation Study 4: Cell Viability Assay for NIH/3T3
Method: NIH/3T3 (mouse embryonic cell line) obtained from American Type Culture Collection (ATCC, Manassas, Va., USA) which used for this assay. Cells were maintained in Dulbecco's medium contains 10% FBS, penicillin (100 U/ml) and streptomycin (100 mg/ml) under humidified atmosphere at 37° C. 2×10⁴ cells seeded per well in 96 well plate followed by 24 hours of incubation.
Treatments of CH formulation were given at different concentrations from 1-10 μg/ml. After 24 hours incubation, MTT (3-(4,5-dimethylthiazol-2-yl)-2,5-diphenyltetrazolium bromide) solution was added per well to achieve a final conc. of 0.5 mg/ml and incubated for 4 hours at 37° C. Isopropanol (solubilizing solution) was added to dissolve formazan crystals and absorbance was recorded at 570 nm on a micro plate reader (Epoch 2 Microplate Reader, BioTek, USA).
Results: As seen in the accompanying FIG. 5, NIH/3T3 cell lines showed no significant effect on cell viability when treated with CH formulation compared to MDA-MB-231 and 4T1. These results make this formulation safe for in vivo experiments.

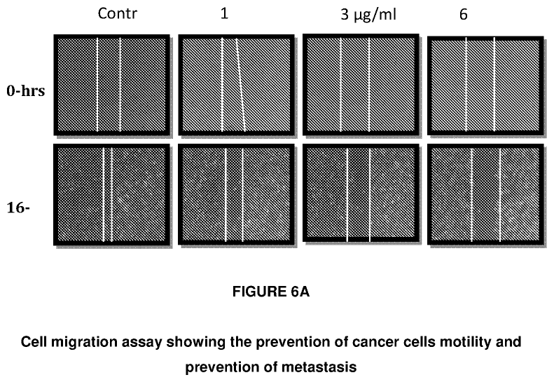

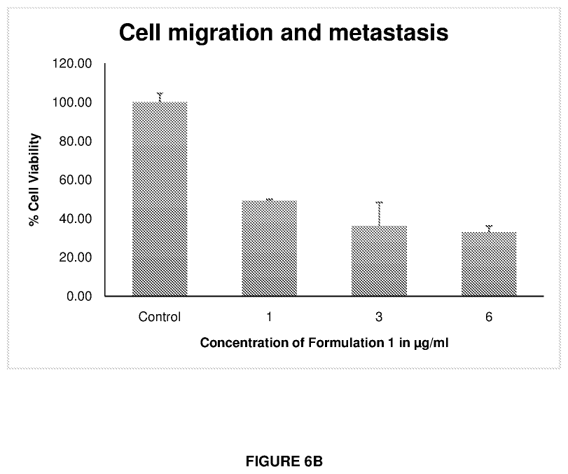

Cell Migration Assay
Cancer cells have tendency to migrate from primary site and metastasize at different tissues and organs which makes it more aggressive and uncontrollable. This assay has been performed to study effect of CH formulation on human breast cancer (MDA-MB-231) cell migration.
To evaluate the effect of formulation 1 cell migration MDA-MB-231 cell line was used. Cells were incubated in 12 well plate to obtain uniform monolayer cell under standard growth conditions. Monolayer was scraped using sterile tip to create wound. Cells were treated with formulation 1 (1 to 6 μg/ml) and incubated for 24 hrs. Formulation 1 inhibited the cell migration by 85-90% at a concentration range 3-6 μg/ml. Observations and results are self-explanatory, as seen in the accompanying FIG. 6A.

Validation Study 5: Wound Healing Assay
Method: Human breast cancer (MDA-MB-231) cell line obtained from American Type Culture Collection (ATCC, Manassas, Va., USA) and used for this assay. MDA-MB-231 cells were maintained in DMEM medium contains 10% FBS, penicillin (100 U/ml) and streptomycin (100 mg/ml) under humidified atmosphere at 37° C. MDA-MB-231 cells were seeded in a 12 well plate and incubated till uniform monolayer achieved.
Sterile T-200 micropipette tip was used to scrape wound in order to make wound of constant width. Cellular debris was removed by DMEM basal medium followed by treatment of CH formulation (1 to 6 μg/ml) and incubated at 37° C. for 24 hrs.
Migration of cells to the wounded area was observed by using phase contrast microscope (Nikon) and photographed (100× magnifications). Distance migrated was measured by image-Pro plus software. Percentage of wound closure was estimated by: Wound closure Percentage=[1−(wound area at Tt/wound area at T0)×100%, where, Tt is 16 hrs after wounding and T0 is the time immediately after wounding.
Results: The results showed significant inhibition of migration of cells with increase in concentrations from 1-6 μg/ml, as shown in the accompanying FIG. 6. Encouraged from observations of above mentioned in-vitro studies, the applicants named herein have proceeded with in-vivo validation as shall be elaborated in the narrative to follow.
For these in-vivo studies, Wistar rats (180-220 g) were used for the study. The animals were maintained under standard laboratory conditions of light-dark cycle (12 h light-12 h dark) and temperature of (22±2° C.) and had free access to food and water.

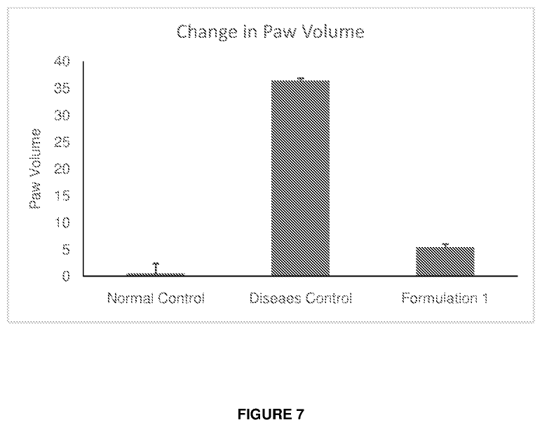

Validation Study 6: Delayed Type Hypersensitivity Assay in SRBC Immunized Rat Model
Method: Wistar rats were treated with test drug and vehicle as described in the experimental protocol. After blood collection, rats were challenged by injection of SRBC (0.5×109 cells/ml/100 g) into the left hind foot pad. Isotonic saline was injected in right hind paw. Paw oedema was measured at 0 and 24 h after SRBCs challenge using digital plethysmometer (Ugobasile, Italy). The pre- and post-challenge difference in the thickness of footpad was expressed in millimeter and specific paw swelling (D %) was calculated which will be taken as a measure of DTH.
Observation: The results of DTH assay showed that CH formulation significantly reduced the paw volume as compared to disease control group indicating an anti-inflammatory action on delayed type hypersensitivity. Effects of CH (CH) formulation on delayed type hypersensitivity assay in SRBC immunized rats can be seen from the accompanying FIG. 7, in which all data is expressed as Mean±SEM.

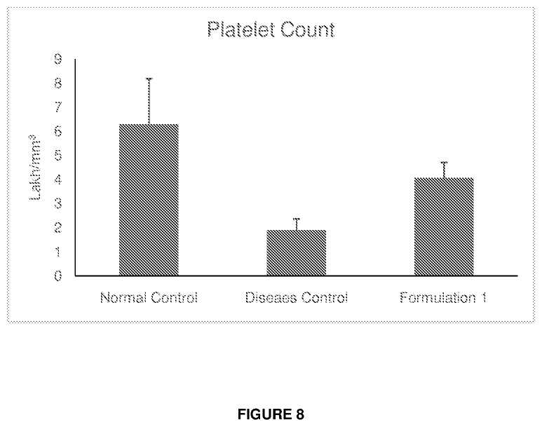

Validation Study 7: Cyclophosphamide Induced Immunosuppression in Mouse Model
Method: Mice were subjected to immunosuppression by hypodermic injection of CPA (70 mg/kg/day) on days 4, 8 and 12 to establish the immunosuppressive animal model, while mice in normal control group was administered with the same volume of sterile physiological saline solution. The animals were treated with test drugs for 14 days. On 14 days, the animals were anesthetized and blood was collected by retro-orbital plexus for platelet count.
Observation: The results showed that CH formulation increased the platelet count as compared to disease control group indicating immunostimulatory effect against cyclophosphamide. Effects of CH (CH) formulation on platelet count in cyclophosphamide suppressed mice can be seen from the accompanying FIG. 8, in which all data is expressed as Mean±SEM.

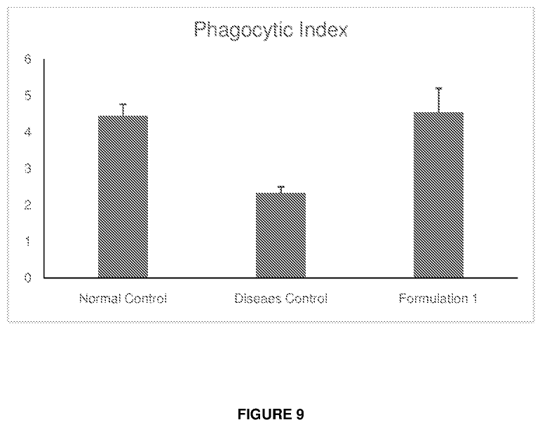

Validation Study 8: Carbon Clearance Test
Method: A carbon clearance test were used to determine the phagocytic index, on day 14, 2 hours after the last administration, mice weighed and then injected with Indian ink (0.1 mL/10 g BW) via the tail vein. A 20 μL specimen of blood were collected from the retro-orbital plexus at 2 and10 min immediately after ink injection. Blood samples mixed with 2 mL of 0.1% Na₂CO₃ solution, and the absorbance measured at 650 nm. Afterwards, mice sacrificed by cervical dislocation. The spleen and liver were excised and immediately weighed. The rate of carbon clearance (K) and the phagocytic index (α) were calculated as follows:

$$K = \frac{lgOD 1 - lgOD 2}{t 2 - t 1} \qquad ( 1 )$$

where t2=10 min, t1=2 min, OD1 and OD2 will be the absorbance at 2 and 10 min, respectively.
Results: Treatment with CH formulation appeared to enhance the phagocytic index by increase in the carbon clearance rate by the cells of reticulo-endothelium system. Effects of CH formulation on phagocytic index in cyclophosphamide suppressed mice can be appreciated from the accompanying FIG. 9 where all data is expressed as Mean±SEM.

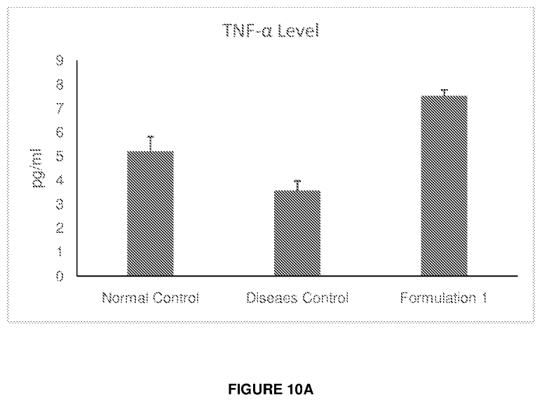

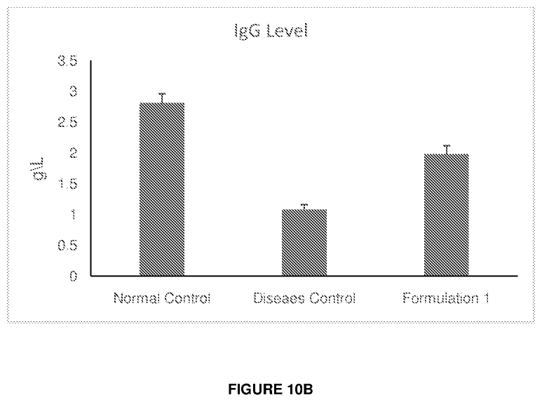

Validation Study 9: TNF-α and IgG Assay
Method: The levels of TNF-α and IgG in serum were analyzed by commercially available ELISA kit, according to the instructions of the manufacturer. The assay was performed by the solid phase sandwich ELISA by adding antigen and biotinylated polyclonal antibodies specific for TNF-α and IgG, to the microtiter plate wells. The plate well walls were precoated with polyclonal antibodies. The HRP conjugated streptoavidin was added and incubated. Further TMB substrate was added to produce the coloured reaction product. The enzyme reaction was stopped by using stop solution. The absorbance of the coloured product was measured using software based microplate reader (ECIL) at 450 nm.
Results: The results showed that treatment with CH formulation increases the levels of TNF-α as compared to disease control indicating the immunostimulatory effect of CH formulation. Effects of CH formulation on TNF-α levels in cyclophosphamide suppressed mice can be seen in the accompanying FIG. 10A, in which all data is expressed as Mean±SEM.
Furthermore, the results showed immunostimulant activity by increasing the antibody IgG in CH treated groups as compared to disease control group. Effects of CH formulation on IgG levels in cyclophosphamide suppressed mice can be seen in the accompanying FIG. 10B, in which all data is expressed as Mean±SEM.

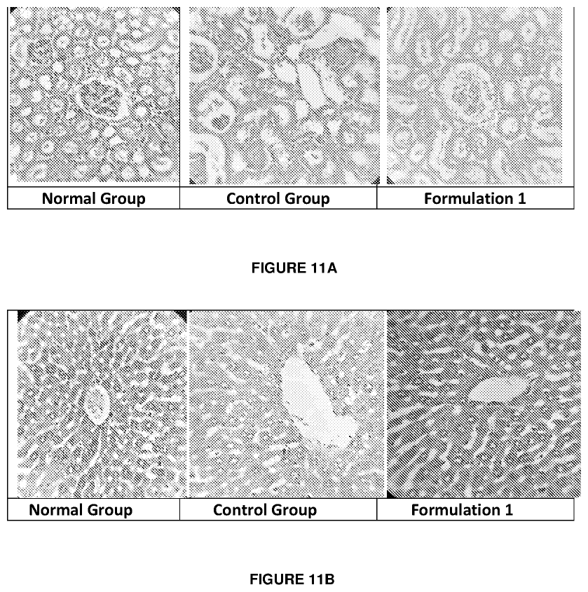

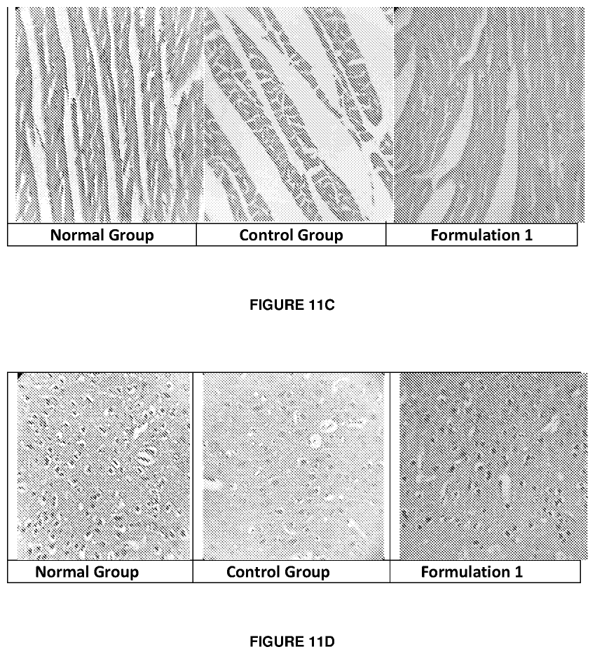

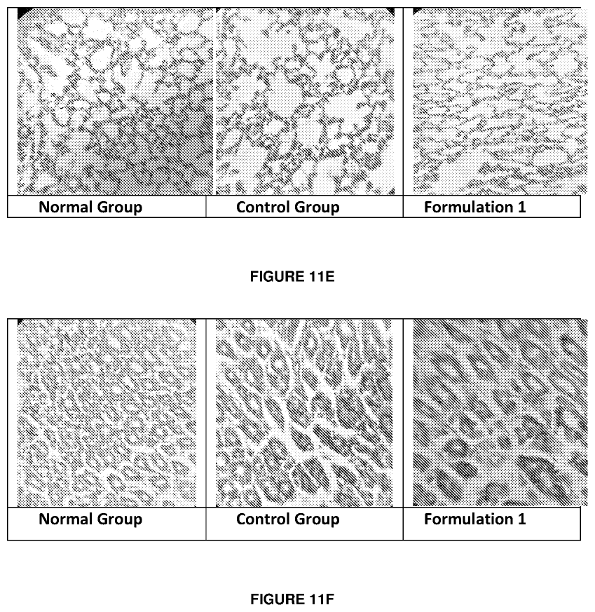

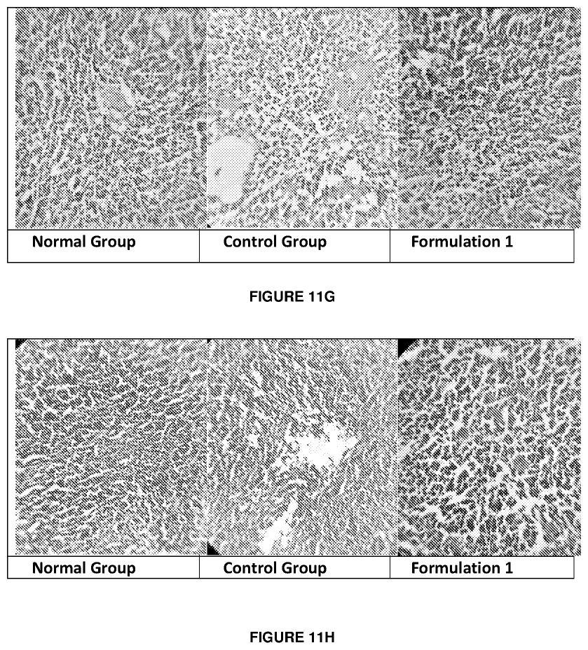

Validation Study 10: Histopathological Studies
Method: The animals were sacrificed by cervical dislocation and the kidney, liver, heart, brain, lungs, stomach and thymus were collected. All organs were fixed with 10% formalin for histopathological analysis. Thin sections of the samples were made using microtome and then stained with hematoxylin and eosin and analyzed under Olympus microscope BX46, Japan, for histological changes.
Results: Referring to the accompanying FIG. 11(A to H), it can be seen that the results showed that normal glomerular structure in normal group when compared to control group. In control group renal tubular damage, hemorrhages, infiltration of neutrophils increase, tubular dilatation, and glomerular atrophy, interruption in the basement membrane around the necrotic tubules and narrowing of the Bowman's space were observed. CH treated group reverses the changes towards normal as compared to control group.
Liver: In positive control group there was completely alerted architecture, centrilobular necrosis, hepatic steatosis, macrovascular and disturbed portal vein architecture noticed. Treatment with CH formulation showed similar changes as normal group.
Heart: In positive control group vacuolization, altered architecture and irregular arrangement, changes in myofibril associated with increased interfibrillar distance and marked infiltration of neutrophils were observed. Treatment with CH formulation reverses these changes towards normal indicating no toxicity of CH formulation.
Brain: In positive control group there was infiltration of neutrophils, increased intracellular space and more vacuoles, density of cells decreased, architecture completely altered and haemorrhage was also observed. Treatment with CH formulation reverses these changes towards normal as in normal group.
Lungs: The positive control group showed hemorrhage, alveolar congestion, thickening of alveolar walls/hyaline membrane formations, and infiltration and aggregation of neutrophils in airspaces or vessel walls as compared to normal group. Treatment with CH formulation showed reduction in inflammatory changes and hemorrhage, thickening of alveolar walls as observed as compared to positive control group.
Stomach: In control group ulceration, hyperemia, bleeding and epithelial cell degeneration and necrosis were observed in the gastric mucosal layer as compared to normal group. Treatment with CH formulation reduces in the ulceration, hyperemia, bleeding and epithelial cell degeneration and necrosis as compared to control group and showed normal structure.
Thymus gland: In control group the cortex becomes thin, thymocytes were small and densely clustered in the cortex. The parenchyma of thymic lobules were atrophied and vascuolized as compared to normal group. Treatment with CH formulation reduced these pathological changes as seen in control group.
Spleen: In control group, parenchymal and capsular fibrosis, lipidosis, splenic necrosis, vacuolization of splenic histiocytes were observed as compared to normal group. Treatment with CH formulation reduced these pathological changes towards normal as compared to control group.

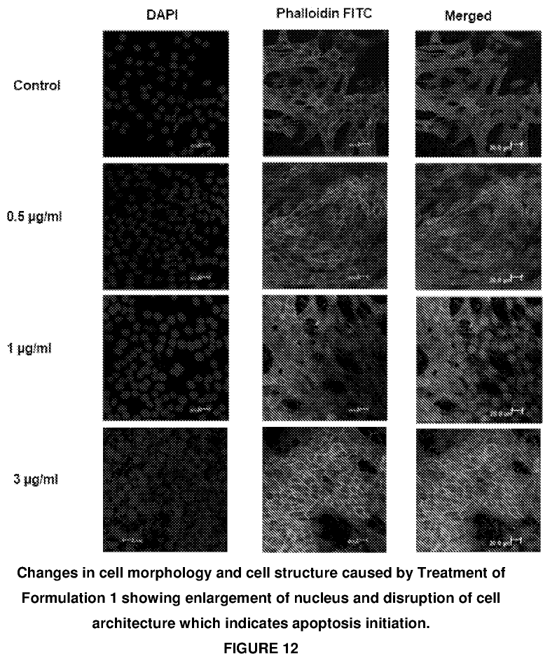

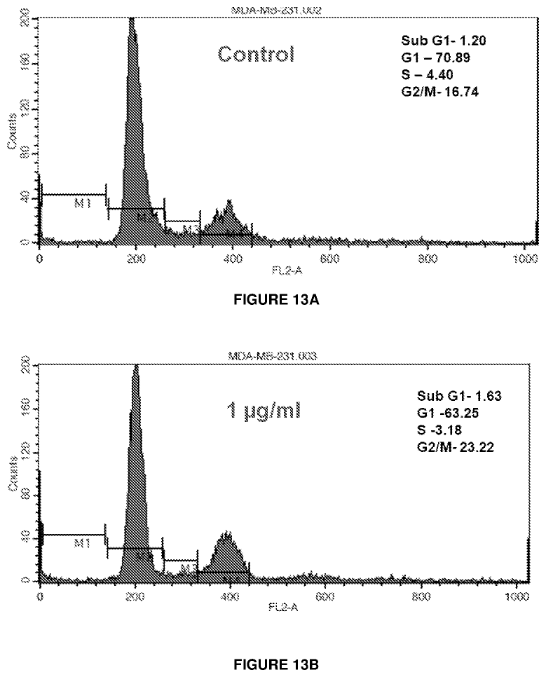

Validation Study 11: Confocal Microscopy Analysis for Actin Depolymerisation
Method: Human breast cancer (MDA-MB-231) cell line obtained from American Type Culture Collection (ATCC, Manassas, Va., USA) and used for this assay. MDA-MB-231 cells were maintained in DMEM medium contains 10% FBS, penicillin (100 U/ml) and streptomycin (100 mg/ml) under humidified atmosphere at 37° C. MDA-MB-231 cells were seeded in a 12 well plate contains glass cover slips and incubated till uniform monolayer achieved. Cellular debris was removed by DMEM basal medium followed by treatment of SH formulation (0.5 to 3 μg/ml) and incubated at 37° C. for 24 hrs. Cells were washed twice with chilled 1× PBS. Fixation of cells had been done with 2% PFA (500 microliter) for 20 minutes at RT. Quenching by 0.1% Glycine (1 mg/ml) (500 microliter) 2-3 minutes at RT. To increase permeability of cells treat it with 0.1% triton X-100 at RT for 10 minutes. Blocking by 1× PBS (10% FBS) at 4° C. for 1 hr or overnight. Phalloidin FITC (1:100 dilutions) added for 45 minutes at RT covered no light. Cells washed twice with chilled 1× PBS. Mounting Media added to each cover slip and sealed it with nail polish. Slides were stored at 4° C. till observation.
MDA-MB-231 cells treated with CH Formulation from 0.5-3 μg/ml for 24 hrs. Cells morphology was observed by using confocal microscope (leica) and photographed (10× magnification).
Results: Fluorescently-labeled phalloidin has binding properties with actin. Higher actin depolymerization and change in cell morphology were observed in Absorf treated cells compared to sorafenib and control. Also, higher actin depolymerisation and change in cell morphology observed in CH treated cells compared to control as seen in the accompanying FIG. 12. CH concentrations creates changes in cell morphology, actin depolymerisation and enlargement of nucleus which are sign of apoptosis.

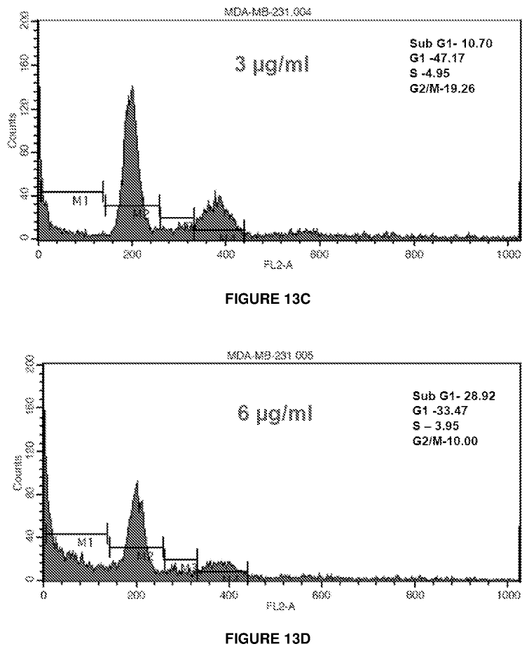

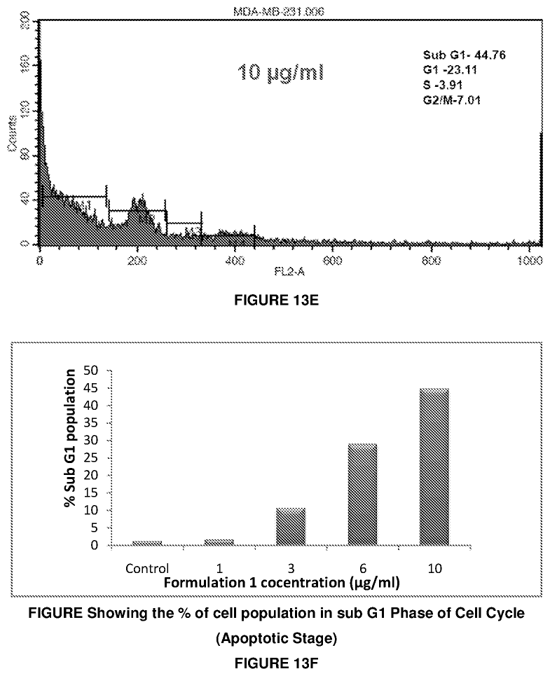

Validation Study 12: Cell Cycle Analysis Through Flow Cytometry Cell Cycle (FACS)
Method: Human breast cancer (MDA-MB-231) cell line obtained from American Type Culture Collection (ATCC, Manassas, Va., USA) which is used for this assay. MDA-MB-231 cells were maintained in DMEM medium contains 10% FBS, penicillin (100 U/ml) and streptomycin (100 mg/ml) under humidified atmosphere at 37° C. Seeding of 2×10⁵ MDAMB-231 cells per well in 60 mm Culture Dish followed by 24 hours of incubation. Treatments of SH formulation (1 to 10 μg/ml) were given for 24 Hours. Cells harvested by trypsinization (including floating cells) in the Centrifugation tube followed by spin down at 3000 RPM for 10 mins at 4° C. and repeat the step by adding ice cold PBS. Fixation of all cells had been done in 70% ethanol at 4° C. overnight.
After this, cells are spin down at 850 g in a centrifuge. Cells treated with ribonuclease (50 μl of a 100 μg/ml sock of RNase). This will ensure only DNA, not RNA, is stained. 450 μl PI (from 50 μg/ml stock solution) were added. Analysis of results done by Measurement of the forward scatter (FS) and side scatter (SS) to identify single cells. Pulse processing is used to exclude cell doublets from the analysis. This can be achieved either by using pulse area vs. pulse width or pulse area vs. pulse height depending on the type of cytometer. PI has a maximum emission of 605 nm so can be measured with a suitable bandpass filter. Filters used are FL2-A.
Results: The results of this study do support a pervious study about the role of CH formulation in inducing cell cycle arrest and apoptosis induction in MDA-MB-231 breast cancer cells. The CH could is promising anticancer formulation against various cancer types.
As seen from the accompanying FIGS. 13(A and B), cell cycle analysis through Flow cytometry cell cycle (FACS) by using Propidium iodide DNA staining on MDA-MB-231 cells treated with SH Formulation from 1-10 μg/ml for 24 hrs can be visualized. Also, histogram represents percentage of cells in different cell cycle phases. (Same data represented in graphical form).

B] Immunomodulation and Other Effects
Immunomodulation and other effects of SP formulation by using in-vitro and in-vivo methods as will be described in detail hereunder.

Validation Study 13: Anti-Oxidant Assay (DPPH Radical Scavenging Activity)
Method: The free radical scavenging activity was measured in terms of hydrogen donating or radical scavenging ability using the stable radical DPPH. 0.1 mM solution of DPPH in methanol was prepared and 1.0 ml of this solution was added to 3.0 ml of different test compounds at different concentrations (6.25-200 μl/ml). After 30 minutes, the absorbance was measured at 517 nm.

$$\% Scavenging = \frac{Absorbance of control - Absorbanc e of test}{Absorbance of control} \times 100 \qquad ( 2 )$$

Formulation was evaluated for antioxidant activity using DPPH inhibition assay and it showed IC₅₀ value of 12.25 μl/ml in comparison to standard ascorbic acid 17.30 μl/ml. Similarly formulation 2 showed hydroxyl radical inhibition at concentration of 29.9 μl/ml in comparison to standard ascorbic acid 30.83 μl/ml. It shows the free radical scavenging protection potential of formulation 2
Observations: The optical density obtained with each concentration of selected test compounds and ascorbic acid was plotted on a graph using the data presented in Table 5 below, taking concentration on X-axis and % inhibition on Y-axis. The graph was extrapolated to find the concentration needed for 50% inhibition and IC₅₀ was calculated.

TABLE 5

| | | | DPPH | | |
|---|---|---|---|---|---|
| | | | radical | Inhibitory | |
| Test | Conc. | Absorbance | inhibition | concentration | |
| compounds | (μl/ml) | (517 nm) | (%) | [IC₅₀] | |
| CH | 3.125 | 1.106 ± 0.0021 | 18.43 | 12.25 | μl/ml |
| Formulation | 6.25 | 0.845 ± 0.0014 | 37.68 | | |
| | 12.5 | 0.578 ± 0.0033 | 57.37 | | |
| | 25 | 0.362 ± 0.0065 | 73.30 | | |
| | 50 | 0.161 ± 0.0035 | 88.12 | | |
| | 100 | 0.092 ± 0.0065 | 93.21 | | |
| | 200 | 0.045 ± 0.0036 | 96.68 | | |
| Ascorbic | 3.125 | 0.688 ± 0.0013 | 29.93 | 17.30 | μg/ml |

| | | | |
|---|---|---|---|
| Acid | 6.25 | 0.4672 ± 0.0018 | 52.42 |
| | 12.5 | 0.282 ± 0.0024 | 71.28 |
| | 25 | 0.119 ± 0.0013 | 87.88 |
| | 50 | 0.078 ± 0.0012 | 92.05 |
| | 100 | 0.049 ± 0.0021 | 95.01 |
| | 200 | 0.688 ± 0.0013 | 29.93 |

Lipid peroxidation in considered as indicator of oxidative degradation of lipid, since formulation 2 showed anti-oxidative. Its action on lipid peroxidation was studied using TBARS method. It showed IC50 value of 22.39 μl/ml which was comparable to positive control 20.32 μl/ml
Results: The results of DPPH assay showed that SP formulation have the proton-donating ability and could serve as free radical inhibitors or scavengers, acting possibly as primary antioxidants. The IC₅₀ value of SP formulation and standard drug ascorbic acid was found to be 12.25 μl/ml and 17.30 μg/ml respectively

Validation Study 14: Hydroxyl Radical Scavenging Activity
Method: Hydroxyl radical is one of the potent reactive oxygen species in the biological system. It reacts with polyunsaturated fatty acid moieties of cell membrane phospholipids and causes damage to cell. Hydroxyl radical scavenging assay was performed by adding 0.1 ml EDTA, 0.1 ml FeCl₃, 0.1 ml H₂O₂, 0.36 ml deoxyribose, 1 ml of test drug (6.25-200 μl/ml), 0.33 ml of phosphate buffer (50 mM, pH 7.4) and 0.1 ml of ascorbic acid in sequence. The mixture was then incubated at 37° C. for 1 hour. 1 ml of the incubated solution was mixed with 1 ml of10% TCA and 1 ml of 0.5% TBA to develop the pink chromogen and the absorbance was measured at 532 nm. The % hydroxyl radical scavenging activity was calculated by the following formula—

$$\% HRSA = \frac{Absorbanceofcontrol - Absorbanceoftestdrug}{Absorbanceofcontrol} \times 100 \qquad ( 3 )$$

where, HRSA is the Hydroxyl Radical Scavenging Activity.
Results: As shown in the Table 6 below, the IC₅₀ value of SP formulation and standard drug ascorbic acid was found to be 29.9 μl/ml and 30.83 μg/ml respectively The results showed that SP formulation have strong scavenging activity of free hydroxyl radicals like hydrogen peroxide, which damages the body cells.

TABLE 6

| | | | TBARS | Inhibitory | |
|---|---|---|---|---|---|
| Test | Conc. | Absorbance | Inhibition | concentration | |
| Compounds | (μl/ml) | (517 nm) | (%) | [IC₅₀] | |
| SH | 6.25 | 0.310 ± 0.122 | 20.32 | 29.9 | μl/ml |
| Formulation | 12.5 | 0.284 ± 0.239 | 27.01 | | |
| | 25 | 0.145 ± 0.431 | 62.73 | | |
| | 50 | 0.104 ± 0.372 | 81.75 | | |
| | 100 | 0.071 ± 0.274 | 87.66 | | |
| | 200 | 0.048 ± 0.314 | 93.61 | | |
| Ascorbic | 6.25 | 0.189 ± 0.132 | 35.27 | 30.83 | μg/ml |

| | | | |
|---|---|---|---|
| Acid | 12.5 | 0.167 ± 0.172 | 42.80 |
| | 25 | 0.135 ± 0.162 | 53.76 |
| | 50 | 0.103 ± 0.183 | 64.72 |
| | 100 | 0.098 ± 0.153 | 64.43 |
| | 200 | 0.068 ± 0.173 | 76.71 |

Validation Study 14: Lipid Peroxidation (TBARS) Inhibition Assay
Method: Malondialdehyde, formed from the breakdown of polyunsaturated fatty acid, serves as a convenient index for determining the extent of peroxidation reaction. Malondialdehyde reacts with thiobarbituric acid (TBA) to form thiobarbituric acid reacting substance (TBARS), a redcolour species, which is measured at 532 nm.
A mixture of 1 ml plant extract, 4 ml ethanol, 4.1 ml of 2.5% linoleic acid in ethanol, 8 ml 0.02M phosphate buffer (pH 7.0) and 3.9 ml distilled water was placed in an oven at 40° C. in the dark for 1 hour. 2 ml of 20% TCA and 2 ml of 0.67% TBA was added to 1 ml of sample solution. The mixture was placed in boiling water bath for 10 minutes. It was centrifuged after cooling at 3000 rpm for 20 minute. The absorbance of the supernatant was measured at552 nm and the % TBARS activity was calculated by the following formula=

$$\% TBARS = \frac{Absorbance of control - Absorbanc e of test}{Absorbance of control} \times 100 \qquad ( 3 )$$

Where TBARS is the thiobarbituric acid reactive substances.
Results: As shown in the Table 7 below, the IC₅₀ value of SP formulation and standard drug ascorbic acid was found to be 22.39 μl/ml and 20.32 μg/ml respectively The results showed the potent lipid peroxidation inhibitory activity of the test drug SP, indicating the anti-oxidant property of SP formulation.

TABLE 7

| | | | TBARS | Inhibitory | |
|---|---|---|---|---|---|
| Test | Conc. | Absorbance | Inhibition | concentration | |
| Compounds | (μl/ml) | (517 nm) | (%) | [IC₅₀] | |
| SH | 6.25 | 0.698 ± 0.219 | 23.29 | 22.39 | μl/ml |
| Formulation | 12.5 | 0.387 ± 0.256 | 57.47 | | |
| | 25 | 0.202 ± 0.191 | 77.80 | | |
| | 50 | 0.123 ± 0.148 | 86.48 | | |
| | 100 | 0.098 ± 0.228 | 89.23 | | |
| | 200 | 0.063 ± 0.261 | 93.07 | | |
| Ascorbic | 6.25 | 0.208 ± 0.167 | 36.0 | 20.32 | μg/ml |

| | | | |
|---|---|---|---|
| Acid | 12.5 | 0.170 ± 0.128 | 47.69 |
| | 25 | 0.147 ± 0.192 | 54.76 |
| | 50 | 0.123 ± 0.232 | 62.15 |
| | 100 | 0.101 ± 0.123 | 68.92 |
| | 200 | 0.089 ± 0.182 | 72.61 |

Encouraged from observations of above mentioned in-vitro studies, the applicants named herein have proceeded with in-vivo validation as shall be elaborated in the narrative to follow.

Animals: Wistar rats (180-220 g) were used for the study. The animals were maintained under standard laboratory conditions of light-dark cycle (12 h light-12 h dark) and temperature of (22±2° C.) and had free access to food and water. The animal care was as per guidelines laid down by the Indian National Science Academy, New Delhi, and the study protocol was approved by the Institutional Animal Ethics Committee, Bagalkot (821/PO/Re/S/2001/CPCSEA).

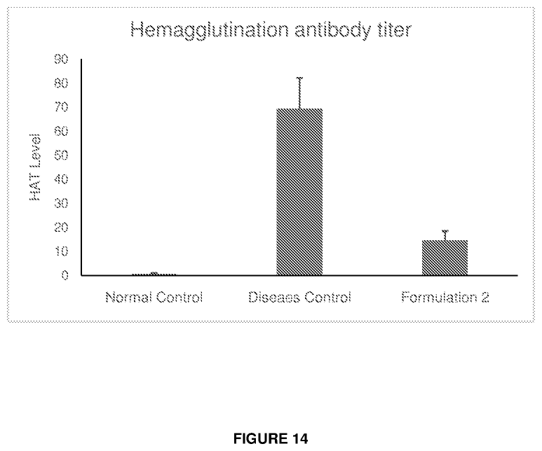

Validation Study 15: Antibody Titer Assay in SRBC Immunized Rat Model
Method: Animals were immunized with sheep red blood cells (SRBC, 0.5×109 cells/ml/100 g) on day 0 then they received vehicle or SP from the day 1 to 5. On the day 6, all rats were anesthetized and blood was collected from the retro-orbital plexus using the microcapillary technique. The serum was assayed for hemagglutination titre was follows: two fold dilutions (0.025 ml) of sera were made in test tubes with saline. To each well 0.025 ml of 1% (v/v) SRBC was added. The test tubes were incubated at 37° C. for 1 h and then observed for hemagglutation by using the double dilution technique. The highest dilution giving haemagglutination was taken as the antibody titer.
Results: The results of antibody titer assay showed that SP formulation possess immunomodulatory effect by controlling the humoral immune response towards normal as compared to the disease control group. Effects of SP formulation on antibody titer assay in SRBC immunized rats is shown in FIG. 14, in which all data is expressed as Mean±SEM.
When formulation 2 was evaluated for its immunomodulation action using rodent model it showed reduction of haemagglutination by 60% in comparison to disease group and normalization of humoral immune response. It also showed decrease in inflammation and attenuates paw volume via delayed type hypersensitivity
Formulation 2 also showed normalization of Platelet count in comparison to disease control group when used in Cyclophosphamide induced immuno-compromised rodent model. Infective agents virus/bacteria survive in body present in body require either activation of cytokine/chemokines for activation of immune

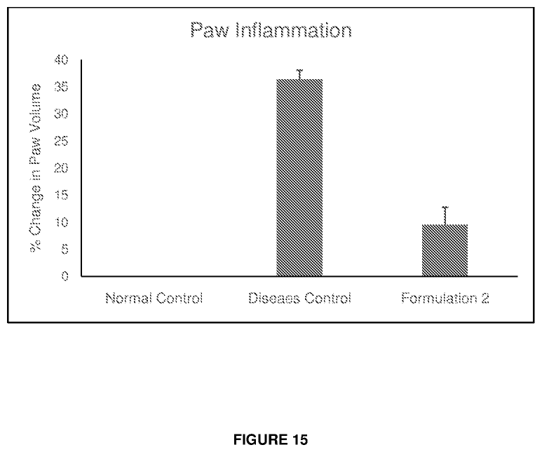

Validation Study 16: Delayed Type Hypersensitivity Assay
Method: Wistar rats were treated with test drug and vehicle as described in the experimental protocol. After blood collection, rats were challenged by injection of SRBC (0.5×10⁹ cells/ml/100 g) into the left hind foot pad. Isotonic saline was injected in right hind paw. Paw oedema was measured at 0 and 24 h after SRBCs challenge using digital plethysmometer (Ugobasile, Italy). The pre- and post-challenge difference in the thickness of footpad was expressed in millimeter and specific paw swelling (D %) was calculated which will be taken as a measure of DTH.
Results: The results of DTH assay showed that SP formulation significantly reduced the paw volume as compared to disease control group indicating an anti-inflammatory action on delayed type hypersensitivity. Effects of SP formulation on delayed type hypersensitivity assay in SRBC immunized rats is shown in FIG. 15, in which all data is expressed as Mean±SEM.
Anti-inflammation mediated delayed type of hypersensitivity protective action of Formulation 1 was evaluated using Paw edema model. Formulation 1 attenuated inflammation and hypersensitivity by 95% in comparison to disease control group.
Formulation 1 also evaluated for efficacy using cyclophosphamide induced thrombocytopenia in rodents and it showed normalization of Platelet count in comparison to disease control group. Formulation will be help in disease caused/chemotherapy induced thrombocytopenia and will help in reducing further complications

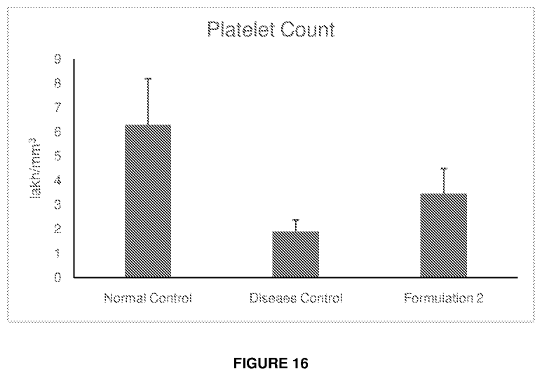

Validation Study 17: Cyclophosphamide Induced Immunosuppression in Mouse Model
Method: Mice were subjected to immunosuppression by hypodermic injection of CPA (70 mg/kg/day)on days 4, 8 and 12 to establish the immunosuppressive animal model, while mice in normal control group was administered with the same volume of sterile physiological saline solution. The animals were treated with test drugs for 14 days. On 14 days, the animals were anesthetized and blood was collected by retro-orbital plexus for platelet count.
Results: The results showed that SP formulation increased the platelet count as compared to disease control group indicating immunostimulatory effect against cyclophosphamide. Effects of SP formulation on platelet count in cyclophosphamide suppressed mice are shown in FIG. 16 in which all data is expressed as Mean±SEM.

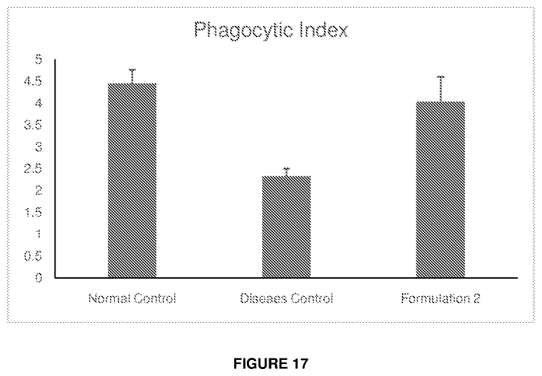

Validation Study 18: Carbon Clearance Test
Method: A carbon clearance test were used to determine the phagocytic index, on day 14, 2 hours after the last administration, mice weighed and then injected with Indian ink (0.1 mL/10 g BW) via the tail vein. A 20 μL specimen of blood were collected from the retro-orbital plexus at 2 and10 min immediately after ink injection. Blood samples mixed with 2 mL of 0.1% Na₂CO₃ solution, and the absorbance measured at 650 nm. Afterwards, mice sacrificed by cervical dislocation. The spleen and liver were excised and immediately weighed. The rate of carbon clearance (K) and the phagocytic index (α) were calculated as follows:

$$K = \frac{lgOD 1 - lgOD 2}{t 2 - t 1} \qquad ( 1 )$$

where t2=10 min, t1=2 min, OD1 and OD2 will be the absorbance at 2 and 10 min, respectively.
Cancer cell survive the defense system either by reducing the humoral immunity or evading the antigen presentation. Activation of phagocytosis and increase in Carbon clearance and Phagocytic index. TNF-α is a pro inflammatory cytokine and play crucial role in control and progression of cancer such as Breast cancer.
Serum immunoglobulin plays important role in cancer progression and low levels has been observed for IgG in breast cancer patients. Treatment of formulation showed increase in serum IgG levels in rodent model.
Formulation 1 treatment showed actin depolymerization and apoptosis induction along with cell cycle arrest potential when evaluated using immunofluorescence and confocal microscopy in MDA-MB-231 Cell line.
Results: Treatment with SP formulation appeared to enhance the phagocytic index by increase in the carbon clearance rate by the cells of reticulo-endothelium system. Effects of SP formulation on phagocytic index in cyclophosphamide suppressed mice can be appreciated from the accompanying FIG. 17 where all data is expressed as Mean±SEM.

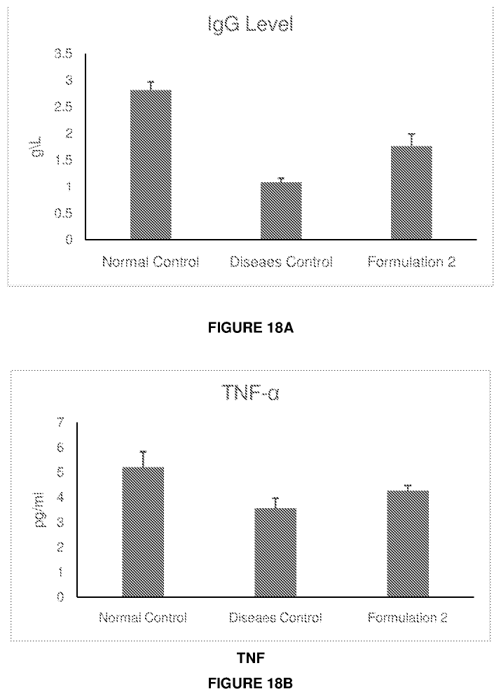

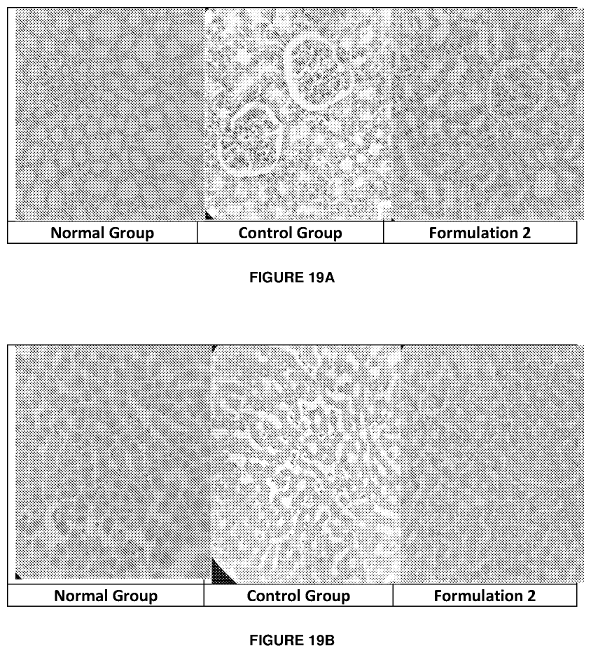

Validation Study 19: Measurement of IgG Levels
Method: The animals were anesthetized and blood will be collected by retro-orbital plexus for the measurement of IgG levels by using Rat IgG (Immunoglobulin G) ELISA Kit.
Results: The results showed immunostimulant activity by increasing the antibody IgG in SP treated groups as compared to disease control group. Effects of SP formulation on IgG levels in cyclophosphamide suppressed mice are shown in FIG. 18 in which all data is expressed as Mean±SEM.

Validation Study 20: Measurement of TNF-α Levels
Method: The levels of TNF-α in serum were analyzed by commercially available ELISA kit, according to the instructions of the manufacturer. The assay was performed by the solid phase sandwich ELISA by adding antigen and biotinylated polyclonal antibodies specific for TNF-α to the microtiter plate wells. The plate well walls were precoated with polyclonal antibodies. The HRP conjugated streptoavidin was added and incubated. Further TMB substrate was added to produce the coloured reaction product. The enzyme reaction was stopped by using stop solution. The absorbance of the coloured product was measured using software based microplate reader (ECIL) at 450 nm.
Results: The results showed that treatment with SP formulation increases the levels of TNF-α as compared to disease control indicating the immunostimulatory effect of SP formulation. Effects of SP formulation on IgG levels in cyclophosphamide suppressed mice are shown in FIG. 18 in which all data is expressed as Mean±SEM.

Validation Study 21: Histopathological Studies

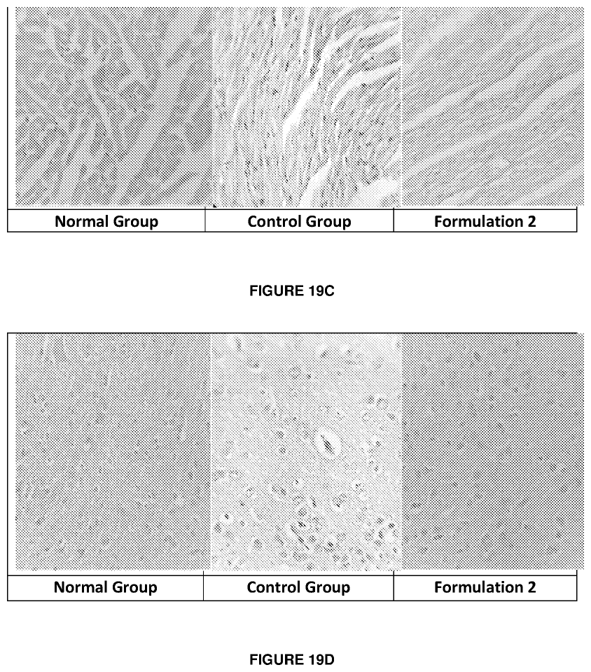

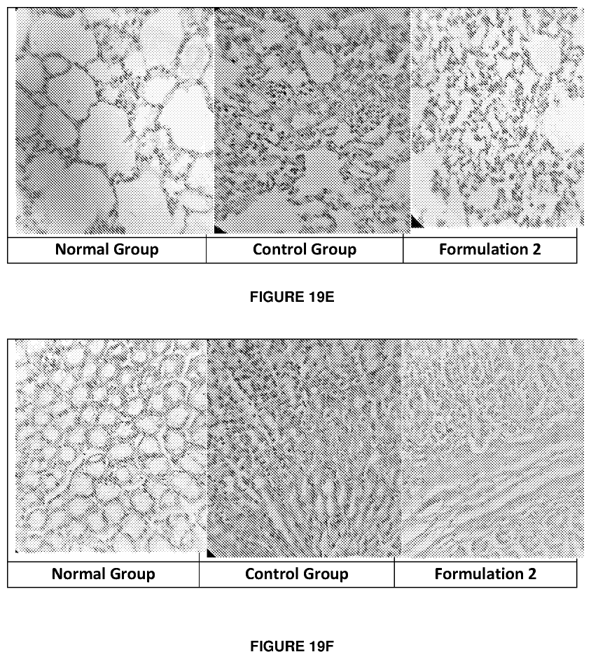

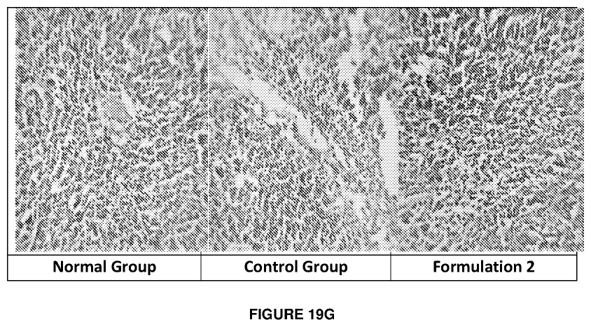

Method:
An acute oral toxicity of formulations 1 has been carried out using Wistar rats. Single oral administration was given to female and male rats up to a dose of 2000 mg/kg. It does not show any mortality or signs of toxicity in animals.
The animals were sacrificed by cervical dislocation and the kidney, liver, heart, brain, lungs, stomach and thymus were collected. All organs were fixed with 10% formalin for histopathological analysis. Thin sections of the samples were made using microtome and then stained with hematoxylin and eosin and analyzed under Olympus microscope BX46, Japan, for histological changes.
Results: During the observation period of 14 days no adverse effect was seen in animals. Gross pathology observation of vital organs showed normal architecture and anatomy. During pharmacological screening of formulations no signs of toxicity or mortality were observed in animals after repeated dosing.
Referring to the accompanying FIG. 19(A to G), following results were seen—in histopathological examination of kidney tissue of normal group showed normal glomerular structure with prominent critical tubules and bowman's capsules. Disease control group showed renal tubular damage, hemorrhages, increased infiltration of neutrophils, tubular dilatation, and glomerular atrophy, interruption in the basement membrane around the necrotic tubules and narrowing of the Bowman's space as compared to normal group. Treatment with SP not produced much difference as compared to disease control indicating that these changes were caused due to SRBC immunization.
The results of histopathological examination of liver showed that in the disease control group there was completely alerted architecture, centrilobular necrosis, hepatic steatosis, macrovascular and disturbed portal vein architecture as compared to normal group. The similar changes were observed after treatment with SP formulation indicating that the SP formulation is not able to control the hepatic damage caused due to immunization and challenge with Sheep RBC. These effects are induced due to SRBC immunization, not by the formulation SP.
The results of histopathological examination of heart showed that the disease control group exhibited vacuolization, altered architecture and irregular arrangement, changes in myofibril associated with increased interfibrillar distance and marked infiltration of neutrophils. Treatment with SP formulation showed no significant difference as compared to control group indicating that the changes were due to SRBC immunization.
The results of histopathological studies of brain tissue showed that in the disease control group there was increased infiltration of neutrophils, intracellular space and increased number of vacuoles, architecture completely altered and also haemorrhages were observed. Treatment with SP formulation showed decreased infiltration of neutrophils and vacuolation and the remaining changes were found to be similar to control group.
The results of histopathological examination of lung tissue showed that in the disease control group inflammatory changes were observed such as hemorrhages, alveolar congestion, thickening of alveolar walls or hyaline membrane formations, and increased infiltration and aggregation of neutrophils in airspaces or vessel walls. Treatment with SP formulation showed reduction in these inflammatory changes as compared to disease control group.
The results of histopathological examination of stomach showed that no histopathological changes were observed in all the groups.
The results of histopathological examination of thymus gland showed that in the positive control group thinning of cortex, small thymocytes densely clustered in the cortex, atrophiedthymic lobules of parenchyma and vacuolization were observed. These pathological changes were reduced after treatment with SP formulation as compared to disease control group.

Validation Study 22: (Safety—In-Vivo Administration)
63 Year Female suffering from carcinoma of esophagus consumed formulation proposed herein at dose of 5 ml thrice a day for 6 months. No residual primary disease lesions were observed after 6 months. Formulation showed no side effects and adverse effects

Validation Study 23: (Antiviral Effect)
Efficacy of formulation 2 was evaluated against SARS-CoV-2 using in vitro assay. It showed 94.5% reduction in viral load within 48 hours of treatment at dose of 1/1024 dilution
Clinical validation of formulation 2 was carried out in SARS-COVID -19 patients to assess the efficacy and safety of the formulation. It reduced the no. of days of hospitalization along with a recovery rate of 96.7% within 7 days of treatment in comparison to 66.7% in Standard treatment group.
Treatment of formulation 2 showed significant increase in levels of IgM on Day 4 similar to standard group but the formulation treatment showed steady state up to 10^{th} day of treatment which was not there in standard treatment group. Whereas IgG levels showed increase after 7^{th} day of treatment Formulation 2 treatment in comparison to standard treatment which showed decrease in IgG levels up to 10^{th} day of treatment.
Formulation 2 also showed significant increase in the levels of mean absolute count of CD4⁺ and CD8⁺ lymphocyte on 7^{th} Day of treatment which and significant increase in absolute count of NK cells (CD16⁺ CD56⁺) and CD19⁺ lymphocytes.
No adverse effects was reported during the clinical evaluation of formulation 2 during the complete clinical trial and all the biochemical parameters were within normal range during the complete study.

The present invention is capable of various other embodiments and that its several components and related details are capable of various alterations, all without departing from the basic concept of the present invention. Modifications and variations of the system and apparatus described herein will be obvious to those skilled in the art. Such modifications and variations are intended to come within ambit of the present invention, which is limited only by the appended claims.

We claim:

1. A synergistic composition, being an admixture of active herbal extract concentrates in particular, having broad spectrum bioactivities, said composition comprising—
0.01% to 15% by weight of Azardiracta indica;
0.01% to 10% by weight of Citrus sinensis;
0.15% to 10% by weight of Zea mays;
0.01% to 10% by weight of Origanum vulgare;
0.01% to 15% by weight of Mentha piperita;
0.01% to 12% by weight of Boswellia serrata;
0.01% to 10% by weight of Citrus bergamia;
0.01% to 15% by weight of Melaleuca alternifloia;
0.01% to 15% by weight of Eucalyptus globules;
0.01% to 15% by weight of Coriandrum sativum;
0.01% to 15% by weight of Rosemarinus officinalis;
0.01% to 15% by weight of Carum copticum;
0.01% to 15% by weight of Nigella sativa;
0.01% to 10% by weight of Cymbopogon citrates;
0.15% to 15% by weight of Saccharum officinarum;
0.02% to 5.0% by weight of Ipomoea aquatic;
0.02% to 5.0% by weight of Eleusine coracana;
0.02% to 5.0% by weight of Biophytum sensitivum;
0.02% to 5.0% by weight of Dioscorea bulbifera;
0.02% to 5.0% by weight of Bambusa bambosa;
0.02% to 5.0% by weight of Taverniera cuneifolia;
0.02% to 5.0% by weight of Camellia sinensis;
0.02% to 5.0% by weight of Vigna vexillata;
0.02% to 5.0% by weight of Delinea indica;
0.02% to 5.0% by weight of Zingiber officinalis;
0.1% to 15.% by weight of Glycyrrhiza glabra;
0.02% to 4.0% by weight of Ocimum sanctum;
0.02% to 5.0% by weight of Lavandulla sp;
0.01% to 10% by weight of Mentha arvensis;
0.01% to 10% by weight of Syzygium aromaticum;
0.01% to 10% by weight of Cinnamon spp. selected between Cinnamon verum and Cinnamon zeylanicum;
0.02% to 5.0% by weight of Cocos nucifera;
Wherein said composition is formulated using standard formulation agents such as Tween 80, PEG, Sugar and thus characterized in—
a) being safe for administration to mammalian subjects; and
b) exhibiting significant broad spectrum bioactivities including anticancer, antiviral, anti-inflammatory and immunomodulatory properties.

2. A method of preparing a synergistic herbal composition exhibiting broad spectrum bioactivities comprising—
a) Selection of raw material, being herbal material in particular;
b) Subjecting the raw material, being herbal material in particular to shade drying;
c) Powdering the shade dried herbs to obtain individual powders;
d) Soaking of the individual powders in solvent to result in herbal solutions;
e) Subjecting the herbal solutions to boiling at 80-100° C. for 60-180 minutes;
f) Subjecting the herbal solutions to concentrating and purification sub-routines to obtain their respective active extract concentrates;
g) Mixing the active extract concentrates amidst stirring at 200 to 1000 RPM to obtain an admixture of active extract concentrates;
h) Sonicating the admixture of active extract concentrates for 15 to 60 minutes for conversion of macromolecules to nano-sized particles;
i) Leaving the sonicated admixture of active extract concentrates to stand for maturation over 24 to 72 hours;
j) Subjecting the matured admixture of active extract concentrates to sonication for 15 to 60 minutes;
k) Adding standard formulation agents such as Tween 80, PEG, Sugar for resulting in a stable formulation of the matured admixture of active extract concentrates.

3. The method of preparing a synergistic herbal composition exhibiting broad spectrum bioactivities as claimed in claim 2, wherein the step of shade drying is carried out over 3 to 7 days.

4. The method of preparing a synergistic herbal composition exhibiting broad spectrum bioactivities as claimed in claim 2, wherein in the step of dissolving the powders in solvents, said components are admixed at a ratio of 1:25 over a period of 24 to 72 hours.

5. The method of preparing a synergistic herbal composition exhibiting broad spectrum bioactivities as claimed in claim 4, wherein the solvents used are selected among the group comprising polar and non polar organic solvents, alcohols, ethyl alcohol and water in particular, their equivalents and their combinations.
